(19) United States
(12) Patent Application Publication
(10) Pub. No.: US 2025/0246053 A1
(43) Pub. Date: Jul. 31, 2025

(54) OUT OF ROOM BED STATUS LIGHT MODULE

(71) Applicant: Hill-Rom Services, Inc., Batesville, IN (US)

(72) Inventors: Aziz Ali Bhai, Fishers, IN (US); Nicholas Comparone, Batesville, IN (US); Unnati Ojha, Apex, NC (US); Vijay Aditya Tadipatri, Madeira, OH (US)

(21) Appl. No.: 19/012,212

(22) Filed: Jan. 7, 2025

Related U.S. Application Data
(60) Provisional application No. 63/626,636, filed on Jan. 30, 2024.

(51) Int. Cl. G08B 5/36; G08B 21/22

(52) U.S. Cl. CPC G08B 5/36; G08B 21/22

(57) ABSTRACT

A device status system includes an outlet positioned in a patient room and operating as a hub for a localized wireless network. A status module is arranged to lie outside the patient room. The status module is connected to the localized wireless network and receives apparatus data over the localized wireless network.

### CROSS-REFERENCE TO RELATED APPLICATIONS

This application claims priority under 35 U.S.C. § 119 (e) to U.S. Provisional Patent Application Ser. No. 63/626,636, filed Jan. 30, 2024, which is expressly incorporated by reference herein.

### BACKGROUND

The present disclosure relates to a bed status module and, in particular, to an out of room bed status module.

Nurse call systems enable communication between caregivers throughout a healthcare facility. Such systems generally provide information about the current status or condition of patients in the facility. Visual indicators are often positioned at various locations throughout a facility to visually notify caregivers when events or conditions relating to a patient occur, so the patient can be attended to in a timely manner. Improved features that are directed to reducing the risk of adverse patient conditions are needed; however, cost is often a concern for the healthcare facility.

For example, nurse call systems generally use several wired connections. In some systems, a bed is connected to a nurse call outlet. The nurse call outlet is then connected via a wired connection to at least one of a nurse station, an indicator light assembly outside the patient room, patient room lighting, a television in the patient room, and/or a central server that manages bed data. Typically, the cost to connect the nurse call outlet to each these remote locations using a wired connection is very high.

### SUMMARY

The present disclosure includes one or more of the features recited in the appended claims and/or the following features which, alone or in any combination, may comprise patentable subject matter.

According to a first aspect of the disclosed embodiments, a device status system includes an outlet positioned in a patient room and operating as a hub for a localized wireless network. The outlet is configured to receive apparatus data from an apparatus connected to the outlet. A status module is arranged to lie outside the patient room. The status module is connected to the localized wireless network. The status module receives the apparatus data over the localized wireless network. Caregivers can view the apparatus data appearing on the status module without entering the patient room.

In some embodiments of the first aspect, the apparatus may be connected to the outlet by inserting a plug of the apparatus into a receptacle of the outlet. The localized wireless network may automatically provision the apparatus to wirelessly connect the apparatus to the localized wireless network in response to the plug of the apparatus being inserted into the receptacle of the outlet. The localized wireless network may automatically provision the apparatus to wirelessly connect the apparatus to the localized wireless network in response to the apparatus being positioned in the patient room. The status module may display apparatus data including at least one of a bed in lo-lo alert, a side rail protocol alert, a bed exit alert, a heart rate alert, and a respiratory rate alert.

Optionally, in the first aspect, a lighting system may be positioned in the patient room and connected to the localized wireless network. The lighting system may be operated in response to apparatus data transmitted over the localized wireless network. The lighting system may be operated to convey a status of the apparatus.

It may be desired, in the first aspect, that at least one indicator light assembly may be positioned outside the patient room and connected to the localized wireless network. The at least one indicator light assembly may be operated in response to apparatus data transmitted over the localized wireless network. The at least one indicator light assembly may be operated to convey a status of the apparatus.

It may be contemplated, in the first aspect, that a display may be coupled to the localized wireless network to display at least a subset of the apparatus data. The apparatus may be a patient support apparatus and the subset of the apparatus data may include at least one of information related to a head of bed angle, a side rail position, a patient alert setting, a patient name, a patient heart rate, a patient respiratory rate, a patient weight, and diagnostic trouble codes. The display may include a control system for controlling the apparatus. The apparatus may be a patient support apparatus and the control system may control at least one of a patient support apparatus lockout, a bed exit alert, a microclimate management system, a heart rate monitor, and a respiratory rate monitor. The display may be static. The display may be mobile. The localized wireless network may automatically provision the display to wirelessly connect the display to the localized wireless network in response to the display moving within a predetermined distance to the patient room. The display may be a touch screen display. The apparatus may be a patient support apparatus.

According to a second aspect of the disclosed embodiments, a patient support apparatus status system includes an outlet positioned in a patient room. The outlet is configured to receive apparatus data from a patient support apparatus connected to the outlet. The patient support apparatus projects patient support apparatus graphical indicia onto the floor to convey at least a first subset of the apparatus data. A bed status module is arranged to lie outside the patient room. The bed status module displays the first subset of apparatus data by illuminating module graphical indicia that matches the patient support apparatus graphical indicia. The module graphical indicia is illuminated in the same color as the support apparatus graphical indicia.

In some embodiments of the second aspect, the patient support apparatus may be connected to the outlet by inserting a plug of the patient support apparatus into a receptacle of the outlet. A localized wireless network may automatically provision the patient support apparatus to wirelessly connect the patient support apparatus to the localized wireless network in response to the plug of the patient support apparatus being inserted into the receptacle of the outlet. A localized wireless network may automatically provision the patient support apparatus to wirelessly connect the patient support apparatus to the localized wireless network in response to the patient support apparatus being positioned in the patient room. The first subset of the apparatus data may include at least one of a bed in lo-lo alert, a side rail protocol alert, a bed exit alert, a heart rate alert, and a respiratory rate alert.

Optionally, in the second aspect, a lighting system may be positioned in the patient room and in communication with the outlet. The lighting system may be operated in response to apparatus data transmitted over a localized wireless network. The lighting system may be operated to convey a status of the patient support apparatus.

It may be contemplated, in the second aspect, that at least one indicator light assembly may be positioned outside the patient room and in communication with the outlet. The at least one indicator light assembly may be operated in response to apparatus data transmitted over a localized wireless network. The at least one indicator light assembly may be operated to convey a status of the patient support apparatus.

It may be desired, in the second aspect, that a display may be coupled to the localized wireless network to display at least a second subset of the apparatus data. The second subset of the apparatus data may include at least one of information related to a head of bed angle, a side rail position, a patient alert setting, a patient name, a patient heart rate, a patient respiratory rate, a patient weight, and diagnostic trouble codes. The display may include a control system for controlling the patient support apparatus. The control system may control at least one of a patient support apparatus lockout, a bed exit alert, a microclimate management system, a heart rate monitor, and a respiratory rate monitor. The display may be static. The display may be mobile. A localized wireless network may automatically provision the display to wirelessly connect the display to the localized wireless network in response to the display moving within a predetermined distance to the patient room. The display may be a touch screen display.

According to a third aspect of the disclosed embodiments, a device status system includes an outlet positioned in a patient room and operating as a hub for a localized wireless network. The outlet is configured to receive apparatus data from an apparatus connected to the outlet. A status module is arranged to lie outside the patient room. The status module is wirelessly connected to the localized wireless network. The status module receives the apparatus data over the localized wireless network. Caregivers can view the apparatus data appearing on the status module without entering the patient room. A lighting system is positioned in the patient room and wirelessly connected to the localized wireless network to convey a status of the apparatus. At least one indicator light assembly is positioned outside the patient room and wirelessly connected to the localized wireless network to convey a status of the apparatus. A display is coupled to the bed status module to display the apparatus data. The display includes a control system for controlling the apparatus.

According to a fourth aspect of the disclosed embodiments, a device status system includes an outlet positioned in a patient room and operating as a hub for a localized wireless network. The outlet is configured to receive apparatus data from an apparatus connected to the outlet. A status module is arranged to lie outside the patient room. The status module is wirelessly connected to the localized wireless network. The status module receives the apparatus data over the localized wireless network. Caregivers can view the apparatus data appearing on the status module without entering the patient room. A lighting system is positioned on the apparatus to convey a status of the apparatus. At least one indicator light assembly is positioned outside the patient room and wirelessly connected to the localized wireless network to convey a status of the apparatus.

In some embodiments of the fourth aspect, the lighting system may shine graphical indicia on floor. The indicator light assembly may provide graphical indicia that matches the graphical indicia provided by lighting system. The statuses may be at least one of a head of bed angle, a side rail position, a patient alert setting, a patient name, a patient heart rate, a patient respiratory rate, a patient weight, and diagnostic trouble codes.

Additional features, which alone or in combination with any other feature(s), such as those listed above and those listed in the claims, may comprise patentable subject matter and will become apparent to those skilled in the art upon consideration of the following detailed description of various embodiments exemplifying the best mode of carrying out the embodiments as presently perceived.

### BRIEF DESCRIPTION OF THE DRAWINGS

The detailed description particularly refers to the accompanying figures in which:

FIG. 1 is a perspective view of an exemplary patient support apparatus in accordance with an embodiment.

FIG. 2 is a schematic view of an exemplary localized wireless network in accordance with an embodiment and including a plurality of smart outlets in communication with medical devices.

FIG. 3 is a schematic view of an exemplary patient support apparatus status system in accordance with an embodiment and including a smart outlet to wirelessly connect various components of the system.

### DETAILED DESCRIPTION

While the concepts of the present disclosure are susceptible to various modifications and alternative forms, specific exemplary embodiments thereof have been shown by way of example in the drawings and will herein be described in detail. It should be understood, however, that there is no intent to limit the concepts of the present disclosure to the particular forms disclosed, but on the contrary, the intention is to cover all modifications, equivalents, and alternatives falling within the spirit and scope of the invention as defined by the appended claims.

The disclosed embodiments eliminate the need to have a wired connection between a nurse call system and an indicator light assembly outside the room, sometimes referred to as a dome light by caregivers and other healthcare professionals, and/or patient room lighting. In the disclosed embodiments, described in more detail below, a central smart outlet is installed in the patient room to act as a hub for a localized wireless network, for example, a Bluetooth network. When the indicator light assembly is installed, the indicator light assembly is paired to the central smart outlet via the localized wireless network. Similarly, the patient room lighting is also paired to the smart outlet via the localized wireless network. In an exemplary embodiment, a bed status module having safety lights is paired to the smart outlet via the localized wireless network. In some embodiments, the safety lights provide alerts, for example, a bed in lo-lo alert, a siderail protocol alert, a bed exit alert, a heart rate alert, or a respiratory rate alert. In an exemplary embodiment, all of the connections are over a Bluetooth mesh so that any device connecting to the hub can have access to the features of the already connected devices.

When a patient support apparatus moves into the patient room, the patient support apparatus is provisioned by the smart outlet. In some embodiments, the provisioning process is automatic. The smart outlet then provides a means for the patient support apparatus to communicate with the patient room lights, the indicator light assembly and the bed status module outside the room.

An out of room touch screen display is also capable of being paired with the smart outlet hub. In some embodiments, the display is static and attached and installed on the wall. In such an embodiment, a service technician pairs the display at the time of install. In some embodiments, the display is mobile. In such an embodiment, as a caregiver moves closer to the patient room, the caregiver utilizes an app on the display to pair the display with one of the available patient rooms. In some embodiments, the display is automatically provisioned to the localized wireless network, when the display is moved into the patient room. In one embodiment the display conveys data related to the patient support apparatus including information related to a head of bed angle, a side rail position, a patient alert setting, a patient name, a patient heart rate, a patient respiratory rate, a patient weight, and diagnostic trouble codes. In some embodiments, the display provides a control system for the patient support apparatus for controlling at least one of a patient support apparatus lockout, a bed exit alert, a microclimate management system, a heart rate monitor, and a respiratory rate monitor. In some embodiments, the patient support apparatus lockout can only be set, and not cleared, with the display. In some embodiments, the bed exit alerts can only be set, and not cleared, with the display. In some embodiments, the heart rate and respiratory rate monitors can only be turned on, and not turned off, with the display.

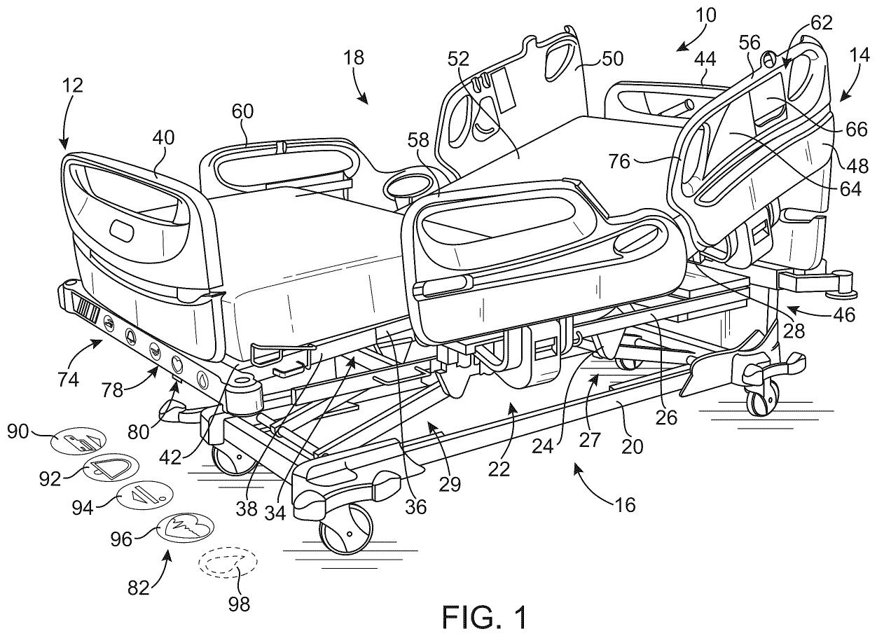

Referring to FIG. 1, a patient support apparatus 10 is illustratively embodied as a hospital bed 10. It will be appreciated that the patient support apparatus 10 is exemplary and the systems described herein are usable with various embodiments of patient support apparatuses. The view shown in FIG. 1 is generally taken from a position that is oriented at the left side, foot end of the hospital bed 10. For purposes of orientation, the discussion of the hospital bed 10 will be based on the orientation of a patient supported on the hospital bed 10 in a supine position. Thus, the foot end 12 of the hospital bed 10 refers to the end nearest the patient's feet when the patient is supported on the hospital bed 10 in the supine position. The hospital bed 10 has a head end 14 opposite the foot end 12. A left side 16 refers to the patient's left when the patient is lying in the hospital bed 10 in a supine position. The right side 18 refers to the patient's right. When reference is made to the longitudinal length of the hospital bed 10, it refers a direction that is represented by the lines that generally extend between the head end 14 and foot end 12 of the hospital bed 10. Similarly, lateral width of the hospital bed 10 refers to a direction that is represented by the lines that generally extend between the left side 16 and right side 18.

The hospital bed 10 includes a base frame 20 which supports a lift system 22. The lift system 22 engages the base and an upper frame 24 such that the lift system 22 moves the upper frame 24 vertically relative to the base frame 20. The lift system 22 includes a head end linkage 27 and a foot end linkage 29. Each of the linkages 27 and 29 are independently operable and may be operated to cause the hospital bed 10 to move into a tilt position which is when the head end 14 of the upper frame 24 is positioned lower than the foot end 12 of the upper frame 24. The hospital bed 10 may also be moved to a reverse tilt position with the foot end 12 of the upper frame 24 is positioned lower than the head end 14 of the upper frame 24.

The upper frame 24 supports a load frame 26. The load frame 26 supports a head deck 28 which is movable relative to the load frame 26. The load frame 26 also supports an articulated seat deck, as is known in the art, also movable relative to the load frame 26 and a fixed seat deck, as is known in the art. Also supported from the load frame 26 is a foot deck 34 that is articulated and moveable relative to the load frame 26. The foot deck 34 in the illustrative embodiment of FIG. 1 provides for powered pivoting of the foot deck 34 and manual extension and retraction of the foot deck 34 to vary the length of the foot deck 34. In other embodiments, powered pivoting of the foot deck 34 may be omitted and the related movement may be caused manually, or follow movement of the articulated seat deck 30. In addition, in some embodiments, extension and retraction of the foot deck 34 may be powered by an actuator.

The foot deck 34 includes a first portion 36 and a second portion 38, which moves relative to the first portion 36 to vary the size of the foot deck 34. The second portion 38 moves generally longitudinally relative to the first portion 36 to vary the longitudinal length of the foot deck 34 and, thereby, the longitudinal length of the hospital bed 10.

A foot panel 40 is supported from the second portion 38 and extends vertically from an upper surface 42 of the second portion 38 to form a barrier at the foot end 12 of the hospital bed 10. A head panel 44 is positioned on an upright structure 46 of the base frame 20 and extends vertically to form a barrier at the head end 14 of the hospital bed 10. A left head siderail 48 is supported from the head deck 28 and is moveable between a raised position shown in FIG. 1 and a lowered position as is known in the art. A right head siderail 50 is also moveable between the raised position of FIG. 1 and lowered position. As shown in FIG. 1, in the raised position, the siderails 48 and 50 extend above an upper surface 52 of a mattress 54 of the hospital bed 10 when the siderails 48 and 50 are in a raised position. In a lowered position an upper edge 56 of the left head siderail 48 is below the upper surface 52.

The hospital bed 10 also includes a left foot siderail 58 and a right foot siderail 60, each of which is supported directly from the load frame 26. Each of the siderails 48, 50, 58, and 60 are operable to be lowered to a position below the upper surface 52. It should be noted that when the head deck 28 is moved, the head siderails 48 and 50 move with the head deck 28 so that they maintain their relative position to the patient. This is because both of the head siderails 48 and 50 are supported by the head deck 28.

A user interface 62 includes a hard panel 64 and a graphical user interface 66. The user interface 62 will be discussed in further detail below, but it should be understood that the hard panel 64 provides indications to a user regarding the status of certain functions of the hospital bed 10 as well as providing a standard set of fixed input devices. The graphical user interface 66 includes a touchscreen display that provides information to a user as well as allowing for flexible, menu driven, operation of certain functions of the hospital bed 10. The graphical user interface 66, also known as a flip-up display (FUD), is mounted to the siderail 48 with a pivotable connection so that the graphical user interface 66 may be pivoted to allow a user the more easily view and interact with the graphical user interface 66, as is known in the art. In some embodiments, the right head siderail 50 may include a second graphical user interface duplicative of the graphical user interface 66.

Additional information is provided to a caregiver through an optional indicator panel 74 which displays the status of various conditions of the hospital bed 10 graphically to a caregiver at the foot end 12 of the hospital bed 10. The location of the indicator panel 74 makes the statuses of the conditions easily discernable from a distance, such that a caregiver may quickly ascertain the statuses from the hallway or the door of a patient's room. As will be discussed below, additional indication of the statuses may be projected on the floor under the foot end 12 of the hospital bed 10, providing larger images on the floor, making the images more easily discerned by a caregiver. Similarly, an illuminated grip 76 is positioned on the left head siderail 48, the illuminated grip 76 being selectively illuminated in different colors to provide an indication of the status of one or more functions of the hospital bed 10 to a caregiver. In some embodiments, the right head siderail 50 also includes an illuminated grip similar to the illuminated grip 76 and which includes the functionality of the illuminated grip 76.

In operation, the grip 76 has four states, not illuminated, illuminated in a blue color, illuminated in an amber color, or illuminated in a red color. In the current embodiment, the grip 76 is not illuminated in one of two conditions: if a patient position monitoring system is disarmed and a patient is in hospital bed 10, or if the patient position monitoring system is armed and the patient is in the proper position in the bed 10. The grip 76 is illuminated blue if the patient position monitoring system is disarmed the patient is out of the hospital bed 10. The blue illumination tends to provide additional lighting for the patient if the ambient light is relatively low. The grip 76 is illuminated in an amber color if the patient position monitoring system is armed and the patient is not in the proper position. This amber illumination provides an additional indication to a caregiver of the alarm condition of the patient position monitoring system. The grip 76 is illuminated in a red color if the patient vital signs monitoring system 100 is in an alarm state, thus, providing an additional indication to a caregiver of the alarm condition of the vital signs monitoring system. An indicator 78 is provided on the foot deck 34 and an illuminator is operable to project patient support apparatus graphical indicia 82 onto the floor beneath the foot deck 34. In some embodiments, the patient support apparatus graphical indicia 82 includes a siderail protocol alert image 90, an alarm setting image 92, a bed in lo-lo alert image 94, and a heart rate alert image 96. Optionally, an incontinence detection alert image 98 is provided. In some embodiments, the images 82 also include a bed exit alert image (not shown).

The caregiver can configure the patient support apparatus 10 to monitor one or more conditions and provide an indication to a caregiver by illuminating the indicator 78 on the foot deck 34, projecting the patient support apparatus graphical indicia 82 on the floor, and/or illuminating the grip 76. In some embodiments, the illumination of the grip 76 in the amber color may be configured to be based on a different condition, such as the expiration of a time between vital signs checks, or any other condition of which the caregiver might need to be reminded. In addition, the illuminated grip may be illuminated in the amber color if any of the alarm conditions of the hospital bed 10 are active, the amber color providing an indication to the caregiver then alarm condition, or a condition that does not meet a patient's care protocol exists. For example, when either the heart rate or respiration rate is within their respective limits, the patient support apparatus 10 displays the heart rate alert image 96 and indicator 78 in a green color. If either the heart rate or respiration rate is outside of an acceptable limit, the patient support apparatus 10 displays the heart rate alert image 96 and indicator 78 in a red color. In some embodiments, the patient support apparatus 10 displays the heart rate alert image 96 and indicator 78 in an amber color if one of the heart rate or respiration rate are approaching an out of limit condition. In some embodiments, the patient support apparatus graphical indicia 82 and indicator 78 may be flashed in the appropriate respective color. In some embodiments, the illuminator 80 may project an image that coincides with the image on the graphical user interface 66 onto the floor so that a caregiver may be able to see the data in real time at a distance.

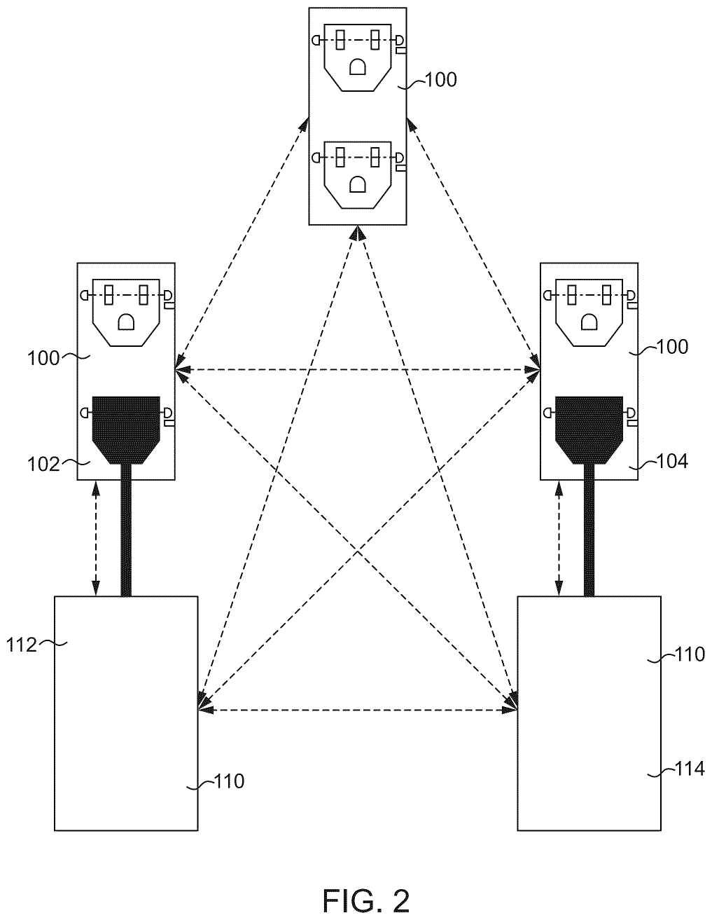

Referring to FIG. 2, a plurality of smart outlets 100 are associated with one another in a medical environment (i.e. a patient room in a healthcare facility automatically provision devices plugged into the smart outlet with a localized network with which the outlet is associated utilizing a time-based method for automatic identification and connection, as described in U.S. Provisional Patent Application Ser. No. 63/594,489, filed Oct. 31, 2023, which is herein incorporated by reference in its entirety). In an exemplary embodiment, the outlets 100 are alternating current (AC) outlets that provide AC power to a room in a healthcare facility. The net effect is that any medical device plugged into a smart outlet 100 will be automatically associated with a localized, wireless network and can communicate on that network with other devices. In some embodiments, the localized wireless network is a Bluetooth mesh network. The Bluetooth mesh network allows for any device 110 in the network to exchange data with another device 110 on the same network. Two devices are not required to be plugged into the same outlet 100 to communicate with one another over the network. Rather, a device 112 plugged into a first outlet 102 can communicate with another device 114 plugged into a second outlet 104, when the first outlet 102 and the second outlet 104 are on the same network. The Bluetooth mesh uses a publish/subscribe model, in one embodiment. It will be appreciated that a model on the network can be a client, server or control model. In some embodiments, groups of smart outlets 100 are configured to communicate over the localized wireless network.

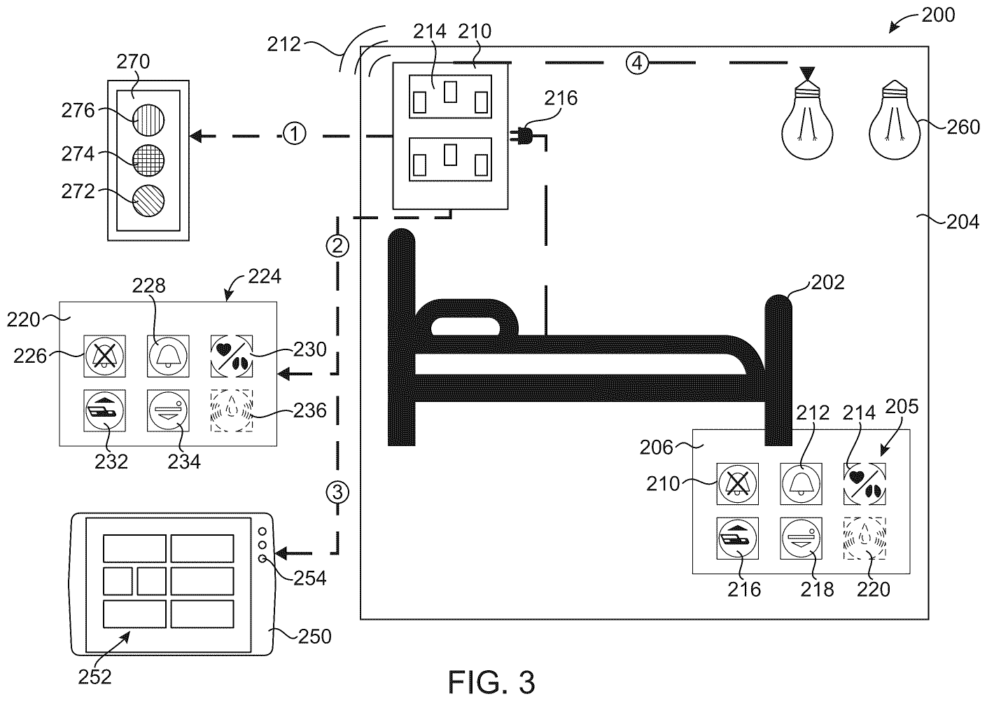

Referring now to FIG. 3, a device status system 200 is operable with a device or apparatus in a facility. In the illustrated embodiment, the device is a medical apparatus in a healthcare facility. More specifically, the device is a patient support apparatus 202 in a patient room 204 of a healthcare facility. In some embodiments, the patient support apparatus 202 is the hospital bed 10 shown in FIG. 1. In an exemplary embodiment, the patient support apparatus 202 includes at least one indicator to display data related to the patient support apparatus 202. For example, in some embodiments, the patient support apparatus 202 includes the indicator 78 shown in FIG. 1 to project one of the images 82 onto the floor. In some embodiments, the patient support apparatus 202 includes a display 206, for example, a display on at least one of the siderails, the footboard, or any other suitable location to display images 208.

In an exemplary embodiment, the images 208 include an alarms deactivated image 210 and an alarms activated image 212. Accordingly, if the alarms associated with the patient support apparatus 202 are activated, the image 212 is illuminated, in some embodiments. If the alarms associated with the patient support apparatus 202 are deactivated, the image 210 is illuminated, in some embodiments. A heart rate and respiratory rate alert image 214 is illuminated, in some embodiments, in response to a measured heart rate and/or respiratory rate. For example, if both the patient's heart rate and respiratory rate are within a normal range, the image 214 is illuminated in a first color, for example, green, in some embodiments. If one of the patient's heart rate and/or respiratory rate is outside of a normal range, the image 214 is illuminated in a second color, for example, yellow, amber or red depending on the severity, in some embodiments. A siderail protocol alert image 216 is illuminated, if the siderail of the patient support apparatus 202 is in a dangerous position. A bed in lo-lo alert image 218 is illuminated, if the deck of the patient support apparatus 202 is in a dangerous position. Optionally, an incontinence detection alert image 220 is illuminated, if an incontinence event is detected. In some embodiments, the images 208 also include a bed exit alert image (not shown) that is illuminated, if the patient exits the patient support apparatus 202.

An outlet 210 (for example, an AC outlet) is positioned in a patient room 204 and is operable to provide power (for example, AC power) to various medical devices in the patient room, including the patient support apparatus 202. In an exemplary embodiment, the outlet 210 operates as a hub for a localized wireless network 212, as described in FIG. 2. For example, in some embodiments, the localized wireless network 212 is a Bluetooth mesh. When an apparatus is plugged into the outlet 210, the outlet 210 is configured to receive apparatus data from an apparatus. For example, when the patient support apparatus 202 is plugged into the outlet 210, the outlet 210 is configured to received patient support apparatus data from the patient support apparatus 202. The patient support apparatus data is then sharable over the localized wireless network 212. That is, the patient support apparatus data is retrievable and usable by any apparatus linked to the localized wireless network 212.

In an exemplary embodiment, the patient support apparatus 202 is connected to the outlet 210 by inserting a plug 216 of the patient support apparatus 202 into a receptacle 214 of the outlet 210. In some embodiments, the localized wireless network automatically provisions the patient support apparatus 202 to wirelessly connect the patient support apparatus 202 to the localized wireless network in response to the plug 216 of the patient support apparatus 202 being inserted into the receptacle 214 of the outlet 210. For example, if the patient support apparatus 202 has never been connected to the localized wireless network 212 of a particular patient room 204, the provisioning process is activated in response to the outlet 210 detecting the plug 216 of the patient support apparatus 202 in the receptacle 214.

In some embodiments, the localized wireless network 212 automatically provisions the patient support apparatus 202 to wirelessly connect the patient support apparatus 202 to the localized wireless network 212 in response to the patient support apparatus 202 being positioned in the patient room 204. For example, in a scenario wherein the patient support apparatus 202 had previously been connected to the localized wireless network 212 of a particular patient room 204, the patient support apparatus 202 is automatically provisioned to the localized wireless network 212, in some embodiments, upon the patient support apparatus 202 being repositioned in the patient room 204. Such a connection is achievable by using a battery function of the patient support apparatus 202 or plugging the patient support apparatus into another outlet not supported by the localized wireless network 212, in some embodiments. Accordingly, once provisioning has occurred between the outlet 210 and the patient support apparatus 202, the patient support apparatus 202 is not required to be plugged into the outlet 210 to link to the localized wireless network 212.

A bed status module 220 is arranged to lie outside the patient room 204. For example, the bed status module 220 is positioned in a hallway outside the door of the patient room 204, in some embodiments. In some embodiments, the bed status module 220 is positioned at a centralized location, such as a nurse's station. The bed status module 220 is connected to the localized wireless network 212 upon installation to receive the patient support apparatus data over the localized wireless network 212, when the patient support apparatus 202 is connected to the localized wireless network 212.

The bed status module 220 communicates the patient support apparatus data to a caregiver without the caregiver entering the patient room 204. The bed status module 220 includes a display 222 that displays module graphical indicia 224 related to the patient support apparatus data. In some embodiments, the module graphical indicia 224 is the same as the patient support apparatus graphical indicia 82. In some embodiments, the module graphical indicia 224 is displayed in the same color as the patient support apparatus graphical indicia 82. In an exemplary embodiment, the module graphical indicia 224 includes an alarms deactivated image 226, and alarms activated image 228, a heart rate and respiratory rate alert image 230, a siderail protocol alert image 232, and a bed in lo-lo alert image 234. Optionally, an incontinence detection alert image 236 is provided. In some embodiments, the module graphical indicia 224 also include a bed exit alert image (not shown).

A caregiver display 250 is connected to and in communication with the bed status module 220. In some embodiments, the caregiver display 250 is static and hardwired to the bed status module 220. For example, in some embodiments, the caregiver display 250 is installed on the wall in the hallway outside the door of the patient room 204 adjacent the bed status module 220. In some embodiments, the caregiver display 250 is installed at a nurse's station. In some embodiments, the caregiver display 250 is mobile and carried by the caregiver. For example, the caregiver display 250 is a tablet or phone, in some embodiments. In such an embodiment, the localized wireless network 212 automatically provisions the caregiver display 250 to wirelessly connect the caregiver display 250 to the localized wireless network 212 in response to the caregiver display 250 moving within a predetermined distance to the patient room 204.

In some embodiments, the caregiver display 250 includes a touch screen display 252. The caregiver display 250 includes various inputs or buttons 254 that enable the caregiver to sort through patient support apparatus data. In some embodiments, the caregiver display 250 displays at least a subset of the patient support apparatus data. In some embodiments, the subset of the patient support apparatus data includes at least one of information related to a head of bed angle, a side rail position, a patient alert setting, a patient name, a patient heart rate, a patient respiratory rate, a patient weight, and diagnostic trouble codes. In some embodiments, the caregiver display 250 includes a control system for controlling the patient support apparatus. In some embodiments, the caregiver operates the various inputs or buttons 254 to direct the control system to control at least one of a patient support apparatus lockout, a bed exit alert, a microclimate management system, a heart rate monitor, and a respiratory rate monitor. Accordingly, a caregiver is capable of reading patient support apparatus data and/or controlling the patient support apparatus 202 without entering the patient room 204.

A lighting system 260 positioned in the patient room 204 and connected to the localized wireless network 212. In some embodiments, the lighting system 260 is wirelessly connected to the localized wireless network 212 upon installation to receive patient support apparatus data from the localized wireless network 212. In some embodiments, the lighting system 260 is operated in response to patient support apparatus data transmitted over the localized wireless network 212 to convey a status of the patient support apparatus 202. For example, the lighting system 260 is turned on or off in response to patient support apparatus data, in some embodiments. The lighting system 260 is configured to blink in response to patient support apparatus data, in some embodiments. In some embodiments, the lighting system 260 is illuminated in a particular color in response to patient support apparatus data. For example, if the patient or patient support apparatus 202 is in need of attention, the lighting system 260 is illuminated is a predetermined color, in some embodiments.

An indicator light assembly 270 positioned outside the patient room 204 and connected to the localized wireless network 212. In some embodiments, the indicator light assembly 270 is wirelessly connected to the localized wireless network 212 upon installation to receive patient support apparatus data from the localized wireless network 212. The indicator light assembly 270 includes a first light 272, a second light 274, and a third light 276, in an exemplary embodiment. In an exemplary embodiment, the first light 272 is green, the second light 274 is yellow or amber, and the third light 276 is red. It will be appreciated that in some embodiments, the indicator light assembly 270 includes and number of lights having any color. In some embodiments, the indicator light assembly 270 is operated in response to patient support apparatus data transmitted over the localized wireless network 212 to convey a status of the patient support apparatus 202. For example, if the patient or patient support apparatus 202 is not in need of attention, the first light 272 is illuminated, in some embodiments. If the patient or patient support apparatus 202 will require attention soon, the second light 274 is illuminated, in some embodiments. If the patient or patient support apparatus 202 is in need of immediate attention, the third light 276 is illuminated, in some embodiments. Accordingly, a caregiver can quickly discern a status of the patient and/or patient support apparatus 202 from outside the patient room.

Any theory, mechanism of operation, proof, or finding stated herein is meant to further enhance understanding of principles of the present disclosure and is not intended to make the present disclosure in any way dependent upon such theory, mechanism of operation, illustrative embodiment, proof, or finding. It should be understood that while the use of the word preferable, preferably or preferred in the description above indicates that the feature so described can be more desirable, it nonetheless cannot be necessary and embodiments lacking the same can be contemplated as within the scope of the disclosure, that scope being defined by the claims that follow.

In reading the claims it is intended that when words such as “a,” “an,” “at least one,” “at least a portion” are used there is no intention to limit the claim to only one item unless specifically stated to the contrary in the claim. When the language “at least a portion” and/or “a portion” is used, the item can include a portion and/or the entire item unless specifically stated to the contrary.

It should be understood that only selected embodiments have been shown and described and that all possible alternatives, modifications, aspects, combinations, principles, variations, and equivalents that come within the spirit of the disclosure as defined herein or by any of the following claims are desired to be protected. While embodiments of the disclosure have been illustrated and described in detail in the drawings and foregoing description, the same are to be considered as illustrative and not intended to be exhaustive or to limit the disclosure to the precise forms disclosed. Additional alternatives, modifications and variations can be apparent to those skilled in the art. Also, while multiple inventive aspects and principles have been presented, they need not be utilized in combination, and many combinations of aspects and principles are possible in light of the various embodiments provided above.

1. A device status system comprising:
an outlet positioned in a patient room and operating as a hub for a localized wireless network, the outlet configured to receive apparatus data from an apparatus connected to the outlet, and
a status module arranged to lie outside the patient room,
wherein the status module is connected to the localized wireless network,
wherein the status module receives the apparatus data over the localized wireless network, and
wherein caregivers can view the apparatus data appearing on the status module without entering the patient room.

2. The system of claim 1, wherein the apparatus is connected to the outlet by inserting a plug of the apparatus into a receptacle of the outlet.

3. The system of claim 2, wherein the localized wireless network automatically provisions the apparatus to wirelessly connect the apparatus to the localized wireless network in response to the plug of the apparatus being inserted into the receptacle of the outlet.

4. The system of claim 1, wherein the localized wireless network automatically provisions the apparatus to wirelessly connect the apparatus to the localized wireless network in response to the apparatus being positioned in the patient room.

5. The system of claim 1, wherein the status module displays apparatus data including at least one of a bed in lo-lo alert, a side rail protocol alert, a bed exit alert, a heart rate alert, and a respiratory rate alert.

6. The system of claim 1, further comprising a lighting system positioned in the patient room and connected to the localized wireless network.

7. The system of claim 6, wherein the lighting system is operated in response to apparatus data transmitted over the localized wireless network.

8. The system of claim 7, wherein the lighting system is operated to convey a status of the apparatus.

9. The system of claim 1, further comprising at least one indicator light assembly positioned outside the patient room and connected to the localized wireless network.

10. The system of claim 9, wherein the at least one indicator light assembly is operated in response to apparatus data transmitted over the localized wireless network.

11. The system of claim 10, wherein the at least one indicator light assembly is operated to convey a status of the apparatus.

12. The system of claim 1, further comprising a display coupled to the localized wireless network to display at least a subset of the apparatus data.

13. The system of claim 12, wherein the apparatus is a patient support apparatus and the subset of the apparatus data includes at least one of information related to a head of bed angle, a side rail position, a patient alert setting, a patient name, a patient heart rate, a patient respiratory rate, a patient weight, and diagnostic trouble codes.

14. The system of claim 12, wherein the display includes a control system for controlling the apparatus.

15. The system of claim 14, wherein the apparatus is a patient support apparatus and the control system controls at least one of a patient support apparatus lockout, a bed exit alert, a microclimate management system, a heart rate monitor, and a respiratory rate monitor.

16. The system of claim 12, wherein the display is static.

17. The system of claim 12, wherein the display is mobile, wherein the localized wireless network automatically provisions the display to wirelessly connect the display to the localized wireless network in response to the display moving within a predetermined distance to the patient room.

18. A patient support apparatus status system comprising:
an outlet positioned in a patient room, the outlet configured to receive apparatus data from a patient support apparatus connected to the outlet, wherein the patient support apparatus projects patient support apparatus graphical indicia onto the floor to convey at least a first subset of the apparatus data, and
a bed status module arranged to lie outside the patient room, wherein the bed status module displays the first subset of apparatus data by illuminating module graphical indicia that matches the patient support apparatus graphical indicia, wherein the module graphical indicia is illuminated in the same color as the support apparatus graphical indicia.

19. A device status system comprising:
an outlet positioned in a patient room and operating as a hub for a localized wireless network, the outlet configured to receive apparatus data from an apparatus connected to the outlet,
a status module arranged to lie outside the patient room, wherein the status module is wirelessly connected to the localized wireless network, wherein the status module receives the apparatus data over the localized wireless network, wherein caregivers can view the apparatus data appearing on the status module without entering the patient room,
a lighting system positioned in the patient room and wirelessly connected to the localized wireless network to convey a status of the apparatus, and
at least one indicator light assembly positioned outside the patient room and wirelessly connected to the localized wireless network to convey a status of the apparatus.

20. The system of claim 19, further comprising a display coupled to the bed status module to display the apparatus data, wherein the display includes a control system for controlling the apparatus.
